(19) United States
(12) Patent Application Publication
(10) Pub. No.: US 2009/0062609 A1
(43) Pub. Date: Mar. 5, 2009

(54) ENDOSCOPE SYSTEM

(75) Inventor: Tadaaki SUDA, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/195,701

(22) Filed: Aug. 21, 2008

(30) Foreign Application Priority Data
Aug. 27, 2007 (JP) 2007-219438

(51) Int. Cl. A61B 1/00

(52) U.S. Cl. 600/117

(57) ABSTRACT

The scope has an imaging sensor, a memory, an image signal-processing unit, and a scope controller having a timer. The image signal-processing unit performs a primary image-processing operation on an image signal output from the imaging sensor. The timer counts an elapsed time. The processor has a video signal-processing unit and a time-hold unit. The video signal-processing unit performs a secondary image-processing operation on the image signal on which the primary image signal processing operation is made. The time-hold unit keeps date data and outputting the date data to the scope controller. The scope controller measures a command-received time when a setting for the primary image-processing operation in the image signal-processing unit is changed on the basis of the elapsed time that is counted by the timer and the date data, and stories a record indicating that the setting has changed and the command-received time to the memory.

### BACKGROUND OF THE INVENTION

1. Field of the Invention

The present invention relates to an endoscope system, and in particular to, an endoscope system whose self-diagnostic operation is easily performed.

2. Description of the Related Art

An endoscope system that has a scope including an imaging sensor is proposed. When a problem occurs in the endoscope system, it is necessary to specify the context of the problem in the endoscope system. Such a problem may include not only a failure of at least one part of the endoscope system, but also an unintended performance of the endoscope system. For example, the case in which an image color does not match the user's intention is cited.

Japanese unexamined patent publication (KOKAI) No. 2004-261612 discloses an endoscope system that has a diagnostic device that performs an error check on each part of the circuit of the image-processing apparatus (the processor).

However, in the case that the color resulting from the image-processing operation is not same as the color that the user intends, causing the user to suspect a problem, the context of the problem can not be determined by the above-mentioned error check function of the endoscope system. And it is hard to specify the context of the problem in the endoscope system.

### SUMMARY OF THE INVENTION

Therefore, an object of the present invention is to provide an endoscope system that stores information which can be a key to analyzing a problem with the scope.

According to the present invention, an endoscope system comprises a scope and a processor. The scope has an imaging sensor, a memory, an image signal-processing unit, and a scope controller having a timer. The image signal-processing unit performs a primary image-processing operation on an image signal output from the imaging sensor. The timer counts an elapsed time. The processor has a video signal-processing unit and a time-hold unit. The video signal-processing unit performs a secondary image-processing operation on the image signal on which the primary image signal processing operation is made. The time-hold unit keeps date data and outputting the date data to the scope controller. The scope controller measures a command-received time when a setting for the primary image-processing operation in the image signal-processing unit is changed on the basis of the elapsed time that is counted by the timer and the date data, and stories a record indicating that the setting has changed and the command-received time to the memory.

### BRIEF DESCRIPTION OF THE DRAWINGS

The objects and advantages of the present invention will be better understood from the following description, with reference to the accompanying drawings in which:

FIG. 1 is a construction diagram of the endoscope system in the embodiment; and

FIG. 2 is a flowchart that shows a process by which the record of the command and the command-received time are stored in the memory.

### DESCRIPTION OF THE PREFERRED EMBODIMENTS

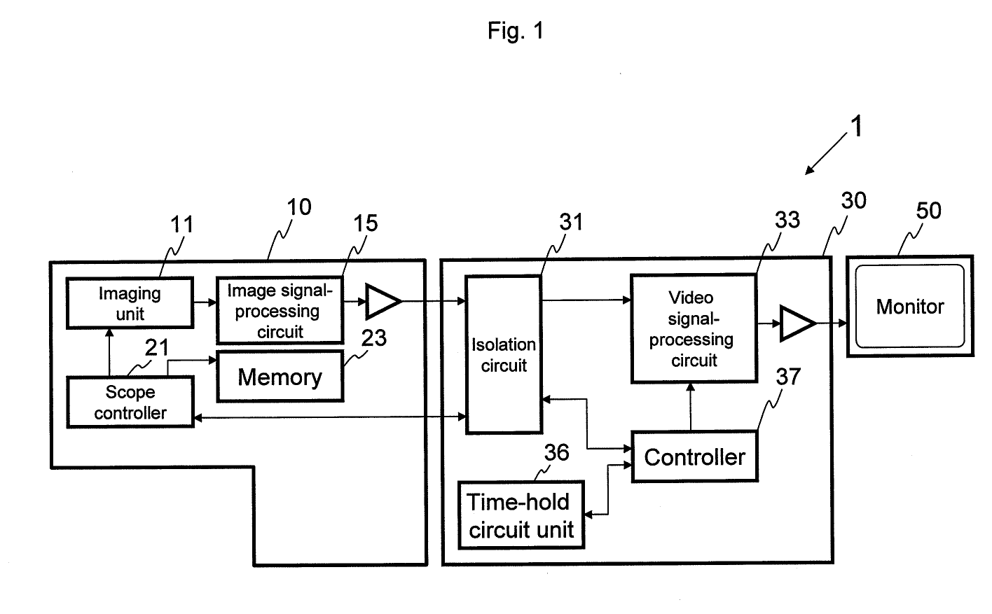

The present invention is described below with reference to the embodiment shown in the drawings. As shown in FIG. 1, the endoscope system 1 in the embodiment comprises a scope 10, a processor 30, and a monitor 50.

The scope 10 has an imaging unit 11 including an imaging sensor such as a CCD, etc., an image signal-processing circuit 15, a scope controller 21, and a memory 23 that is non-volatile.

An imaging unit 11 captures an image and outputs an image signal based on the captured image to the image signal-processing circuit 15. The image signal-processing circuit 15 performs a primary image-processing operation including a YC separation, etc., on the image signal output from the imaging unit 11, and then outputs the image signal to the processor 30.

The processor 30 has an isolation circuit 31, a video signal-processing circuit 33, a time-hold circuit unit 36, and a controller 37.

The processor 30 performs a secondary image-processing operation that generates a video signal to display the video image on the monitor 50 on the basis of the image signal output from the image signal-processing circuit 15.

The monitor 50 is connected to the processor 30. The monitor 50 is a display device that displays an image compatible with the predetermined video format.

Furthermore, another device, such as an external storage device that stores the data of the video signal, etc., and a printer that prints out the image based on the video signal, may be connected to the processor 30.

Next, the details of each part of the endoscope system 1 are explained.

The imaging sensor of the imaging unit 11 receives the reflected light (or the exciting light) from the photographing subject through the objective optical system (not depicted).

The optical image of the photographing subject on the incident surface of the imaging sensor is captured, the photoelectric conversion is performed, and then the image signal based on the optical image is output to the image signal-processing circuit 15.

The primary image-processing operation on the image signal output from the imaging unit 11 is made by the image signal-processing circuit 15. Then, the image signal after the primary image-processing operation is output to the video signal-processing circuit 33 through the isolation circuit 31.

The secondary image-processing operation on the image signal output from the image signal-processing circuit 15 is made by the video signal-processing circuit 33 so that a video signal for displaying the image on the monitor 50 is generated.

The time-hold circuit unit 36 has a clock function that counts the date and hour and keeps the date and hour data.

In addition, the time-hold circuit unit 36 has a power supply for backup, such as a battery, etc. Therefore, when the AC power supply that supplies power to each part of the processor 30 is set to the OFF state, the power supply for backup supplies power to the time-hold circuit unit 36 so that the clock function of the time-hold circuit unit 36 is continuously performed.

In the embodiment, when the scope 10 is connected to the processor 30 and then the endoscope system 1 is set to the ON state, in other words, the power supply to the endoscope system 1 by the AC power supply commences, the scope controller 21 receives the date and hour data from the time-hold circuit unit 36 through the controller 37.

In the embodiment, the date and hour is counted by the clock function and the power supply for backup in the time-hold circuit unit 36, however, the date and hour may be obtained by another method. For example, the time-hold circuit unit 36 may be connected to the internet through a network and may obtain the time from a time server on the internet.

The scope controller 21 includes a timer that counts an elapsed time from the date and hour indicated by the date and hour data received from the time-hold circuit unit 36.

The scope controller 21 measures a command-received time when the scope controller 21 has received a command for changing the settings for the primary image-processing operation, such as brightness, etc., in the image signal-processing circuit 15, on the basis of the time elapsed from the date and hour.

A record of the command and the command-received time are stored in the memory 23, in order to allow read-out by a PC, etc., that is connected with the scope 10 directly or through the processor 30.

Specifically, the scope 10 has an interface such as RS-232 (Recommended Standard 232), etc., that can be connected with an external device such as a PC, etc. Through the interface, the record of the command and the command-received time are read out to the external device.

Furthermore, the interface which is used for connecting the external device may be used for connecting between the scope 10 and the processor 30.

In the case that the PC is connected with the scope 10 through the processor 30, the processor 30 also has an interface such as RS-232, etc., that can be connected with the external device such as the PC, etc.

The record of the command and the command-received time are read out from the memory 23 to the PC, etc., and they are used in a diagnostic operation to trace the commands of the scope 10, in case a problem occurs in the scope 10. A problem may include not only a failure of at least one part of the endoscope system 1, but also an unintended performance of the endoscope system 1, such as an image color not matching the user's intention.

The settings for the primary image-processing operation in the image signal-processing circuit 15 are not usually changed by the user of the endoscope system 1. However, they may be accidentally changed by the user or intentionally during the course of maintenance.

When the settings for the image-processing operation are changed by these operations, the color displayed on the monitor 50, etc., may be incorrect. In this case, the user may request that the endoscope system 1 be serviced, due to suspicion of a problem.

However, it is hard to specify the context of the problem in the endoscope system 1, because it is not an error of the endoscope system 1 but merely a change in the settings.

In the embodiment, the change tracking of the command of the scope 10 is stored in the memory 23 with the command-received time, so that these can be read out to the PC, etc. Information including the record of the command and the command-received time can be a key to analyze the problem in the scope 10. Therefore, the service personnel can easily identify the context with using the information.

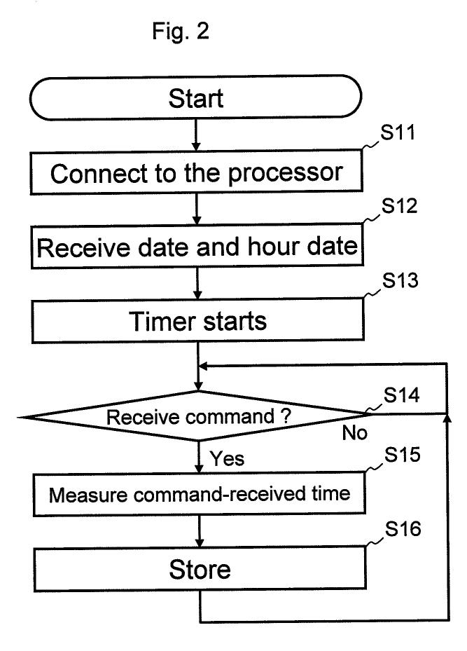

Next, the process that stores the record of the command and the command-received time in the memory 23 is explained with reference to the flowchart in FIG. 2.

In step S11, the scope 10 is connected to the processor 30 and then the endoscope system 1 is set to the ON state, in other words, the power supply to the endoscope system by the AC power supply commences. In step S12, the date and hour data is transmitted to the scope controller 21 from the time-hold circuit unit 36 through the controller 37 and the isolation circuit 31.

In step S13, the scope controller 21 starts the timer so that it counts the elapsed time from the date and hour recorded in the date and hour data.

In step S14, it is determined whether the operation for changing the settings for the primary image-processing operation in the image signal-processing circuit 15 was performed indicating that the scope controller 21 received the command corresponding to this operation.

When it is determined that the scope has received the command corresponding to the operation for changing the settings for the primary image-processing operation, the operation continues to step S15, otherwise, step S14 is repeated.

In step S15, the scope controller 21 measures (reads) the command-received time when the scope controller 21 received the command for changing the settings for the primary image-processing operation, on the basis of the time elapsed from the recorded date and hour.

In step S16, the record of the command and the command-received time are stored in the memory 23, so that these can be read out by the PC that is connected to the scope 10 or the processor 30.

Then, the operation is returned to step S14 so that the operations in steps S14 to S16 are repeated while the endoscope system 1 is set to the ON state.

Although the embodiment of the present invention has been described herein with reference to the accompanying drawings, obviously many modifications and changes may be made by those skilled in this art without departing from the scope of the invention.

The present disclosure relates to subject matter contained in Japanese Patent Application No. 2007-219438 (filed on Aug. 27, 2007) which is expressly incorporated herein by reference, in its entirety.

1. An endoscope system comprising:
a scope that has an imaging sensor, a memory, an image signal-processing unit, and a scope controller having a timer, said image signal-processing unit performing a primary image-processing operation on an image signal output from said imaging sensor, said timer counting an elapsed time; and
a processor that has a video signal-processing unit and a time-hold unit, said video signal-processing unit performing a secondary image-processing operation on said image signal on which said primary image signal processing operation is made, said time-hold unit keeping date data and outputting said date data to said scope controller;
said scope controller measuring a command-received time when a setting for said primary image-processing operation in said image signal-processing unit is changed on the basis of the elapsed time that is counted by said timer and said date data, and storing a record indicating that said setting has changed and said command-received time to said memory.

2. The endoscope system according to claim 1, wherein said scope controller stores said record and said command-received time, so that said record and said command-received time can be read out by an external device that is connected with at least one of said scope or said processor.
